(19) United States
(12) Patent Application Publication
(10) Pub. No.: US 2024/0252100 A1
(43) Pub. Date: Aug. 1, 2024

(54) PROSTATE MEASUREMENT SYSTEM

(71) Applicant: MedicaMetrix, Inc., Lowell, MA (US)

(72) Inventors: Shalmali Nitin Salunke, Somerville, MA (US); Peter Pythagoras Leonidas Carayannopoulos, Winchester, MA (US); Robert F. Rioux, Ashland, MA (US)

(21) Appl. No.: 18/427,093

(22) Filed: Jan. 30, 2024

Related U.S. Application Data
(60) Provisional application No. 63/442,042, filed on Jan. 30, 2023.

(51) Int. Cl. A61B 5/00

(52) U.S. Cl. CPC A61B 5/4381

(57) ABSTRACT

The present invention is directed to a prostate measurement system including an improved medical assembly allowing for the accurate measurement of a patient's prostate to be subsequently used in determining whether the patient is at increased risk of having prostate cancer or other prostate-related issues.

### CROSS-REFERENCE TO RELATED APPLICATIONS

This application claims the benefit of, and priority to, U.S. Provisional Patent Application No. 63/442,042, filed Jan. 30, 2023, the content of which is hereby incorporated by reference in its entirety.

### TECHNICAL FIELD

The disclosure relates generally to a system for measuring and/or evaluating a body part. More specifically, the present invention is directed to a prostate measurement system including an improved medical assembly allowing for the accurate measurement of a patient's prostate to be subsequently used in determining whether the patient is at increased risk of having prostate cancer or other prostate-related issues.

### BACKGROUND

Prostate-related problems are widespread in the male population, especially the older male population. In particular, the prostate gland is prone to three main conditions, including: prostatitis (infection or inflammation of the prostate); benign prostatic hyperplasia (BPH) (aging-related enlargement of the prostate gland); and prostate cancer (the growth of cancerous cells inside the prostate, which may break out of the gland and affect other parts of the body). In fact, other than skin cancer, prostate cancer is the most common cancer in men in the U.S., and is also the second-leading cause of cancer death, after lung cancer.

While the chance of survival improves significantly if prostate cancer is detected early and treated effectively, conventional detection systems and methods for detecting prostate problems, including cancer, are still lacking. For example, while ultrasound systems have been developed to diagnose prostate problems, such systems are very expensive. Most ultrasound imaging is performed by radiologists at an outside facility, or at the practitioner's office on a contract basis with a portable ultrasound unit. The technology and interpretation is difficult to master, requiring a time-consuming learning curve. Consequently, no routine examining system or technique exists which provides a high degree of accuracy in measuring prostate volume, nor is the required repeatability of results achieved.

Thus, the digital rectal examination (DRE) continues to be the modality of choice for monitoring the prostate, even though the process is very subjective. The standard digital rectal examination is performed by inserting a finger into a patient's rectum and palpating or feeling the palpable surface of the prostate. The physical characteristics of the prostate size, contour, consistency, symmetry, and the presence or absence of nodularity, are assessed and recorded by attempting to translate the physician's subjective impressions into a written record. This method of data collection is inexact and makes comparisons from exam to exam very difficult.

### SUMMARY

The present invention is directed to a prostate measurement system including an improved medical assembly allowing for a physician to obtain an accurate measurement of a patient's prostate via a digital rectal examination (DRE) procedure. Upon utilizing the medical assembly to obtain measurements of dimensions of the prostate, the system is able to apply a proprietary algorithm to the collected prostate measurements and automatically determine a prostate volume. A medical professional (i.e., physician, clinician, etc.) can then use the calculated prostate volume to provide a more accurate assessment of a patient's prostate health. In particular, the accurate prostate volume measurement provided by the system of the present invention enables physicians to assess a patient's prostate status to better plan and monitor treatments, opt for drug therapies, and determine data driven recommendations for prostate biopsies versus active surveillance.

The system includes a measurement assembly configured to be worn upon a medical professional's finger and used to measure dimensions of a patient's prostate. For example, the measurement assembly may generally be in the form of a finger probe. The medical professional, while wearing the assembly upon their finger, can advance the assembly into the patient's rectum and perform a conventional DRE with the use of the measurement assembly, allowing for the collection of measurement data by the assembly. In particular, the measurement assembly, upon being swiped across the palpable surface of the prostate, is configured to collect measurement data and subsequently transfer such data to a control/console unit operably coupled to the measurement system. The control/console unit is configured to receive and process said measurement data, including determining or estimating various properties of the prostate, such as the palpable surface width of the prostate or the volume of the prostate, for example. The system further includes a disposable barrier configured to be removably placed over the measurement assembly. The disposable barrier acts as a sterile layer of protection between the anatomy (i.e., the patient's rectum) and the measurement assembly, which ultimately maintains the measurement assembly in a clean state during use. The disposable barrier provides sufficient protection while maintaining the necessary tactile feedback to the operator when using the measurement assembly, particularly when swiping the finger probe across the prostate surface.

Accordingly, the prostate measurement system of the present invention provides a minimally invasive medical device designed to accurately measure the volume of the prostate gland early in the diagnostic process and throughout a patient's ongoing care with his clinical team. The measurement assembly (i.e., fingertip probe) and disposable barrier are designed for simple, intuitive operation by a urologist, urologic oncologist, urological nurse, or the like (i.e., operation of the device simply requires that the user have a working knowledge of prostate anatomy, of the DRE procedure, and that the user can reliably identify the lateral margins of the prostate gland by palpation). In turn, the prostate measurement system enables early evaluation of prostate health through accurate volumetric measurements, differentiating patients at higher risk and mitigating uncertainty. Patients ultimately benefit from the system of the present invention by avoiding invasive, higher-risk procedures and having better care management, which leads to a better quality of life.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 is a block diagram of a prostate measurement system consistent with the present disclosure.

FIG. 2 is perspective view of an exemplary control/console unit coupled to an exemplary measurement assembly consistent with the present disclosure.

FIG. 3 is a perspective view of an exemplary measurement assembly, generally in the form of a fingertip probe, consistent with the present disclosure.

FIG. 4 is a side view of an exemplary disposable barrier configured to be removably placed over a measurement assembly consistent with the present disclosure.

FIG. 5 is a top view of an exemplary disposable barrier consistent with the present disclosure, illustrating various dimensions (i.e., various lengths of the barrier and an inner diameter) of at least the distal portion of the barrier to be placed over the measurement assembly and accommodate operation thereof.

FIGS. 6A and 6B are top and side views of an exemplary disposable barrier consistent with the present disclosure, illustrating at least the inner dimensions of the distal portion of the barrier.

FIG. 7 is a top view of an exemplary disposable barrier consistent with the present disclosure including an insert provided within an interior of the sleeve portion of the barrier and adjacent to the distal portion so as to improve the ease with which an operator can slid their hand into and out of the barrier.

FIG. 8 shows an operator interacting with the measurement assembly, specifically wearing the fingertip probe upon their finger and the disposable barrier placed over the fingertip probe and extending over the operator's hand so as to provide a sterile barrier during the DRE procedure.

FIG. 9 is a top view of another embodiment of a disposable barrier consistent with the present disclosure.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present invention is directed to a prostate measurement system that includes an improved medical assembly allowing for the accurate measurement of a patient's prostate to be subsequently used in determining whether the patient is at increased risk of having prostate cancer or other prostate-related issues. The system of the present invention enables early evaluation of prostate health through accurate volumetric measurements, differentiating patients at higher risk and mitigating uncertainty. Patients benefit by avoiding invasive, higher-risk procedures and having better care management, which leads to a better quality of life.

As previously described herein, the prostate gland is prone to three main conditions, including: prostatitis (infection or inflammation of the prostate); benign prostatic hyperplasia (BPH) (aging-related enlargement of the prostate gland); and prostate cancer (the growth of cancerous cells inside the prostate, which may break out of the gland and affect other parts of the body).

Prostate volume can be used early in the prostate cancer diagnostic process to calculate the Prostate Specific Antigen Density (PSAD), which is an enhanced biomarker for the detection of prostate cancer. PSAD allows clinicians to more accurately identify patients at risk and direct future diagnostic tests and treatments. This can minimize the need for painful biopsies and non-essential prostate cancer treatments that can have adverse effects for patients.

BPH is a common condition for men as they age. Prostate volume gives the physician an indication of the likely progression of BPH symptoms over time. Prostate volume helps guide and direct treatment options for BPH. For example, TURP and UROLift typically are successful over a specific range of volumes. Alpha blockers work better in combination with finasterides, but only if the prostate is >40 cc.

Male lower urinary tract symptoms (MLUTS) symptoms include incomplete voiding and/or chronic urinary retention symptoms. The severity of MLUTS is best measured using quantitative indicators such as overall prostate volume. Increased prostate volume is a significant risk factor for disease progression in men who have MLUTS due to BPH. Approximately 50-60% of men with chronic prostatitis experience troublesome MLUTS. These symptoms tend to have a more substantial impact on patient quality of life than other MLUTS presentations. The effects of MLUTS become more pronounced for prostatitis patients with >50 cc prostate volume.

The prostate measurement system of the present invention allows for a physician to measure prostate volume early in the diagnostic process, and, in conjunction with other tools, enables more accurate diagnosis of prostate health and differentiates patient risk, allowing clinicians to make appropriate and effective treatment decisions. For example, the volumetric prostate measurement in combination with PSA further characterizes risk through PSAD, to improve sensitivity and specificity. The system of the present invention can provide clinician decisions for therapies that are more appropriately targeted to a prostate patient's condition. Furthermore, the higher accuracy provided by the system of the present invention results in a reduction of painful, debilitating and costly treatments for patients. The system assists clinicians throughout the entire prostate health journey to effectively monitor and continually reassess and stratify patient risk in continuum of care.

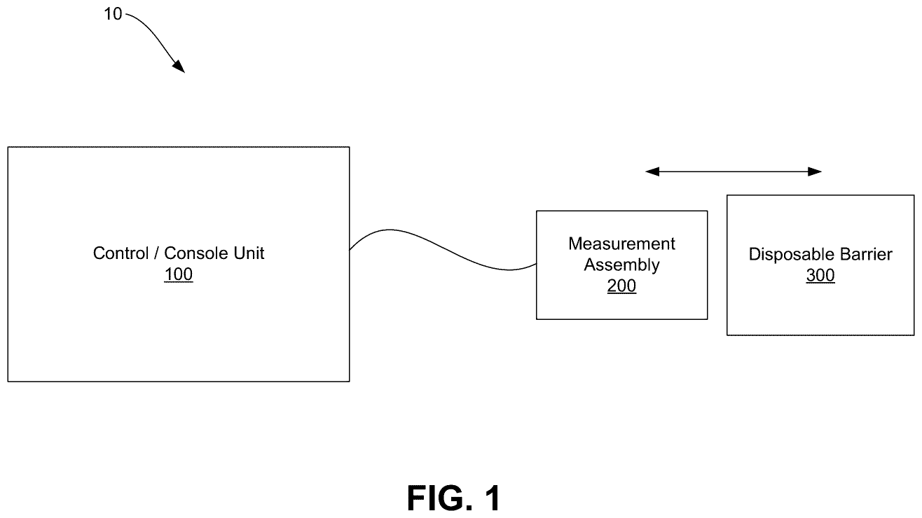

FIG. 1 is a block diagram of a prostate measurement system 10 consistent with the present disclosure. The system generally includes a control/console unit 100, a measurement assembly 200 configured to be operably coupled to the control/console unit 100 (via a cable or the like) and configured to obtain measurements of the prostate, and a disposable barrier 300 shaped and/or sized to be placed over the measurement assembly 200 during use thereof.

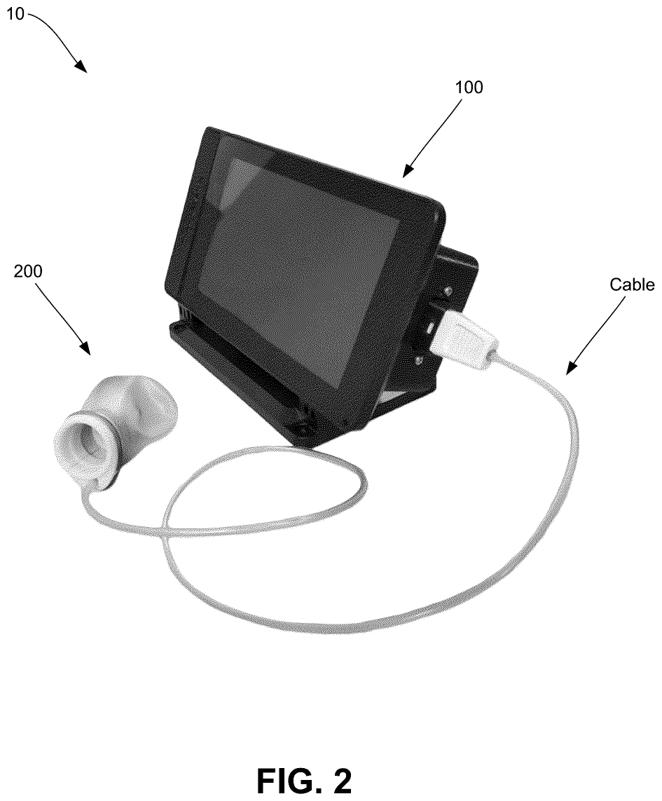

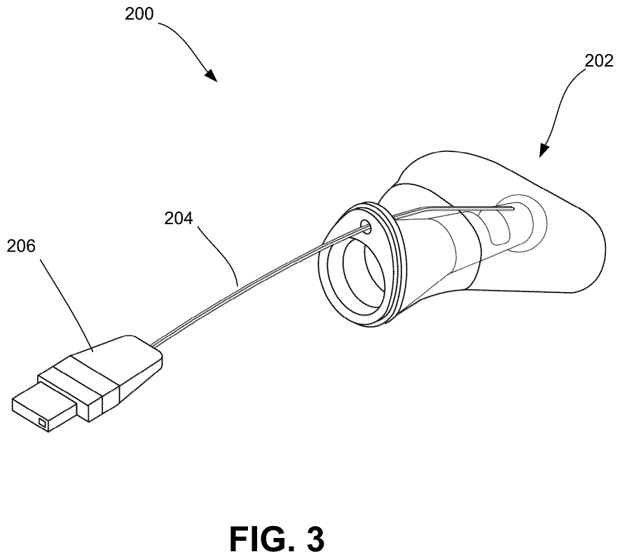

FIG. 2 is perspective view of an exemplary control/console unit 100 coupled to an exemplary measurement 200 assembly consistent with the present disclosure. FIG. 3 is a perspective view of an exemplary measurement assembly 200, generally in the form of a fingertip probe, consistent with the present disclosure.

As will be described in greater detail herein, the measurement assembly 200 is configured to provide information indicative of a dimension of the object of interest (i.e., the prostate) to the control/console unit 100. The control/console unit 100 is configured to estimate one or more properties or conditions of the prostate based on the information provided by the measurement assembly 200.

As shown, the measurement assembly 200 may generally include a probe portion 202 generally configured to be worn upon a medical professional's finger and used to measure dimensions of a patient's prostate. The medical professional, while wearing the fingertip probe portion 202 upon their finger, can advance the probe 202 into the patient's rectum and perform a conventional DRE procedure with the use of the measurement assembly, allowing for the collection of measurement data by the assembly 200. In particular, the probe 202, upon being swiped across the palpable surface of the prostate, is configured to collect measurement data and subsequently transfer such data to the control/console unit 100 operably coupled to the measurement assembly 200 via a cable 204 (air and/or fiber optic cable(s)) extending from the probe 202 and coupled to the control/console unit 100 via a connector 206.

In turn, the control/console unit 100 is configured to receive and process said measurement data, including determining or estimating various properties of the prostate, such as the palpable surface width of the prostate or the volume of the prostate, for example.

The control/console unit 100 and the measurement assembly 200 may generally include the various prostate measuring components and designs, including examination devices, probes, and control unit hardware, as described in U.S. Pat. Nos. 7,309,319; 8,694,079; 8,838,214; 9,402,547; 9,402,564; 9,538,952; and 11,638,552, the contents of each of which are incorporated by reference herein in their entireties.

For example, in the illustrated embodiment, the measurement assembly 200 includes, for example, a finger clip having a roller ring or wheel rotatably mounted thereto and disposed within an inflatable membrane. The roller ring can include a measurement pattern positioned opposite to optical fibers configured to receive light reflected from the measurement pattern. A user can put on the finger clip, position the membrane in proximity to a rectal wall overlying a prostate, and inflate the membrane. As the user slides their finger across the inside of the membrane, which is pressed against the rectal wall, the roller ring can rotate with respect to the fibers such that the fibers move relative to the measurement pattern. In turn, the control/console unit 100 can sense light reflected through the fibers from the reference pattern and calculate or estimate various attributes of the prostate based on the reflected light, including, but not limited to, palpable surface width of the prostate or the volume of the prostate. The control/console unit 100 includes a durable hardware device which contains the various sensors, electronics, air pump, valves, and a display to support the operation of the measurement assembly 200 and further transmit the optical signal (i.e., light returning through the fiberoptics from the calibrated grid) into an electronic signal and hence into useful units of measurement, for the subsequent calculation of prostate volume via a proprietary algorithm, and further display the results to the user.

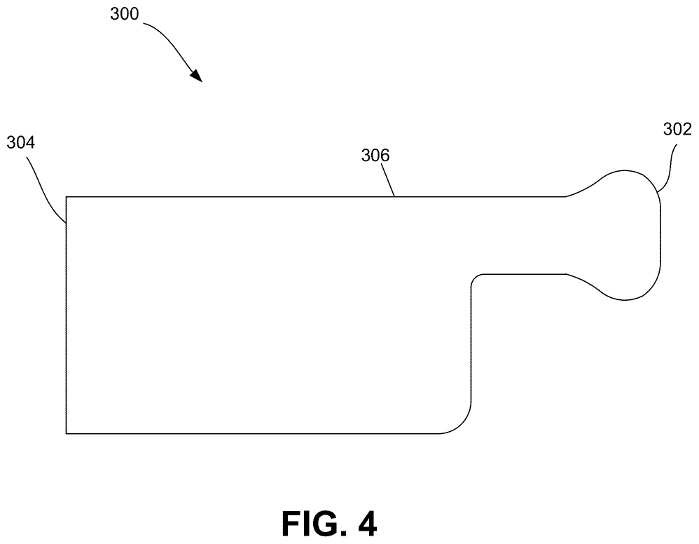

The system 10 further includes a disposable barrier, such as a cover or sock, configured to be removably placed over the measurement assembly 200. FIG. 4 is a side view of an exemplary disposable barrier 300 configured to be removably placed over the fingertip probe 202. As shown, the barrier 300 may generally include a closed distal end 302, configured to be placed over, and completely surrounding, the fingertip probe 202, and an open proximal end 304 allowing for the barrier to be removably placed over the fingertip probe and to further accommodate a user's hand, including a user's finger upon which the fingertip probe 202 of the measurement assembly 200 is mounted during a procedure. The barrier 300 further includes a sleeve portion 306 of sufficient length so as to at least fully cover a user's hand and to further cover the cable 204 extending from the fingertip probe 202 of the measurement assembly 200.

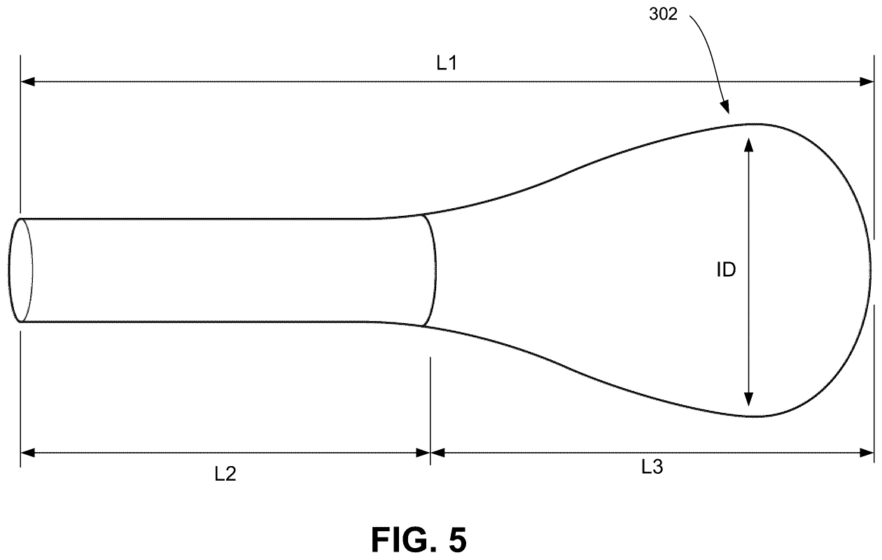

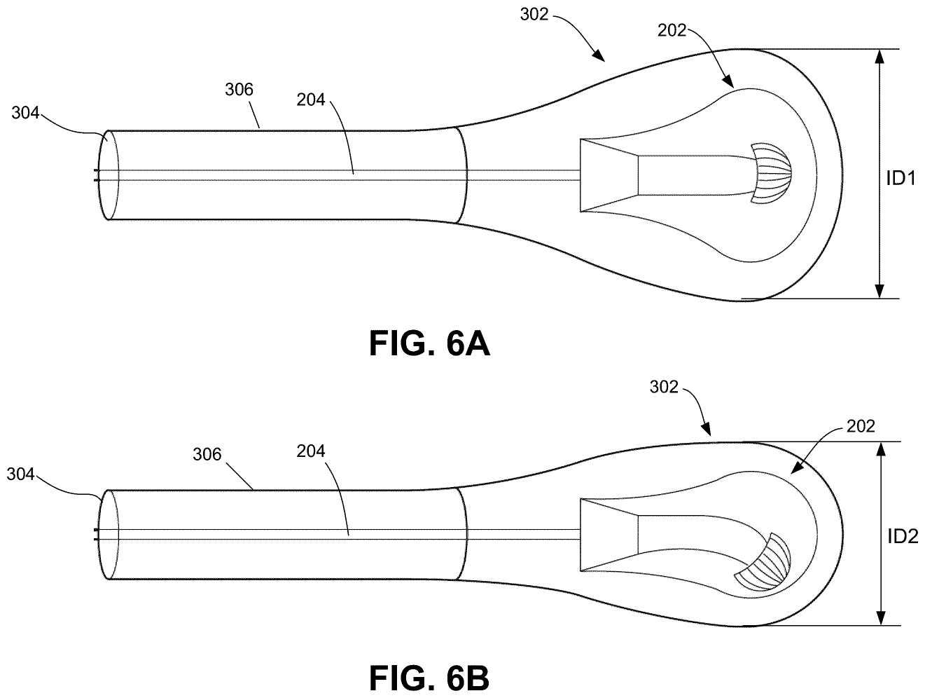

FIG. 5 is a top view of the exemplary disposable barrier 300, illustrating various dimensions (i.e., various lengths of the barrier 300 and an inner diameter (ID)) of at least the distal end 302 to be placed over the fingertip probe 202 portion of the measurement assembly 200 and accommodate operation thereof. FIGS. 6A and 6B are top and side views of an exemplary disposable barrier 300 disposed over the fingertip probe portion 202 of the measurement assembly 200, illustrating inner dimensions of the distal end 302 of the barrier 300.

Referring to FIG. 5, the barrier 300 may have an overall length (L1) of about 90 cm, for example. It should be noted that the overall length (L1) may be greater than 90 cm (i.e., 91 cm, 92, cm, 93 cm, 94 cm, 95 cm, 96 cm, 97 cm, 98 cm, 99 cm, 100 cm, etc.) or less than 90 cm (i.e., 89 cm, 88 cm, 87 cm, 86 cm, 85 cm, 84 cm, 83 cm, 82 cm, 81 cm, 80 cm, etc.). The sleeve portion 306 of the barrier 300 may have a length (L2) sufficient to fully accommodate and cover at least a portion of a user's hand and full cover cable(s) extending from the fingertip probe. For example, in one embodiment, the length (L2) of the sleeve 306 may be approximately 65 cm. It should be noted that the sleeve length (L2) may be more or less than 65 cm. The closed distal portion 302 of the barrier 300 may have a length (L3) sufficient to fully accommodate and cover the fingertip probe portion 202 of the measurement assembly 200. For example, in one embodiment, the length (L3) of the closed distal end 302 may be approximately 25 cm. It should be noted that the distal end length (L3) may be more or less than 25 cm.

The closed distal end 302 of the barrier 300 has an inner diameter (ID) that is sufficiently sized and/or shaped so as to fully accommodate the fingertip probe during use, including fully accommodate the probe when any portion thereof is fully inflated. For example, as described in U.S. Pat. No. 11,638,552, the measurement assembly includes a membrane that, when the user is ready to take a measurement, the membrane can be positioned adjacent to the rectal wall in proximity to the prostate and then inflated such that the membrane expands into contact with the rectal wall. As such, the inner diameter (ID) of the distal end 302 of the barrier 300 is at least greater than the outer diameter of the fingertip probe portion 202 when in a fully inflated state.

For example, as shown in in FIGS. 6A and 6B, when fully inflated, the fingertip probe portion 202 may be approximately 100 mm in width and approximately 40 mm in height. Accordingly, a first inner diameter (ID1) of the closed distal end 302 of the barrier 300 is at least 101 mm (width direction) and a second inner diameter (ID2) of the closed distal end 302 is at least 41 mm (height direction).

The disposable barrier 300 acts as a sterile layer of protection between the anatomy (i.e., the patient's rectum) and the fingertip probe 202 of the measurement assembly 200, which ultimately maintains the fingertip probe 202 of the measurement assembly 200 in a clean state during use. The disposable barrier 300 provides sufficient protection while maintaining the necessary tactile feedback to the operator when using the fingertip probe 202, particularly when swiping the fingertip probe 202 across the prostate surface. As previously described herein, the barrier 300 is sufficiently fitted over the fingertip probe 202, particularly when the member of the fingertip probe 202 is in an inflated state, to thereby allow for the user to achieve an accurate measurement. If there are wrinkles after the membrane has been inflated, then inaccurate measurements can occur. Accordingly, the barrier cannot be too snug, or there will be a risk that that barrier 300 will tear when the membrane is inflated. The barrier 300 may be comprised of a durable material capable of withstanding pressure placed upon it during a DRE procedure, but flexible enough to allow for tactile feedback when a user is swiping the fingertip probe 202 across the prostate surface during the procedure. Furthermore, the barrier 300 is preferably transparent (to allow for a user to have visibility when pulling the barrier 300 on and off during use). Furthermore, the barrier 300 may include a non-stick material. For example, in some embodiments, the barrier 300 may be comprised of a material or may include a coating that exhibits non-stick properties.

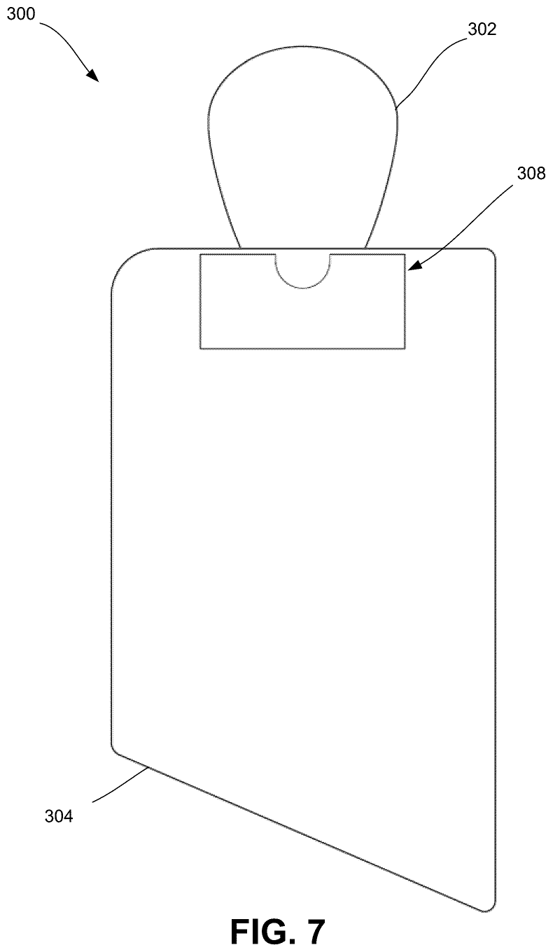

FIG. 7 is a top view of an exemplary disposable barrier consistent with the present disclosure including an insert 308 provided within an interior of the sleeve portion of the barrier and adjacent to the distal portion so as to improve the ease with which an operator can slide their finger and fingertip probe 202 into and out of the barrier 300. For example, the material from which the barrier 300 is made may have a tendency to stick to itself as a result of static cling and/or the inherent stickiness of the material itself.

The insert 308 may generally be comprised of a rigid material configured to maintain a desired shape and provide sufficient structure within an interior of the barrier 300 so as to prevent opposing interior surfaces of the barrier 300 from contacting and thus sticking to one another. Accordingly, the insert 308 improves the ease with which a user can slide their hand into and out of the interior of the barrier 300 by essentially preventing interior surfaces of opposing portions of the barrier 300 from contacting and sticking to one another.

For example, a user may first mount the fingertip probe 202 onto a finger, and then slide their finger into the interior of the barrier 300. The insert 308 is positioned within the sleeve portion 306 and adjacent to the distal end 302 and is shaped and/or sized so as to maintain separation of opposing interior surfaces of the barrier 300 at or near the entry point into the distal end 302. The insert 308 further includes a cutout, void, and/or aperture that is generally centered with the distal end 302 and is shaped and/or sized to allow for a user's finger (along with the fingertip probe 202 mounted thereto) to pass therethrough and into the distal end 302. As such, the insert 308 effectively centers and provides a guide for a user's finger into the distal end 302 so as to avoid any unintentional contact with an interior surface of the barrier 300 until the user's finger with the fingertip probe 202 is sufficiently advanced into the distal end 302. Without the insert 308, it can be challenging for a user to slide their finger and the fingertip probe 202 into the distal end 302 without sticking to the interior surface of the barrier 300.

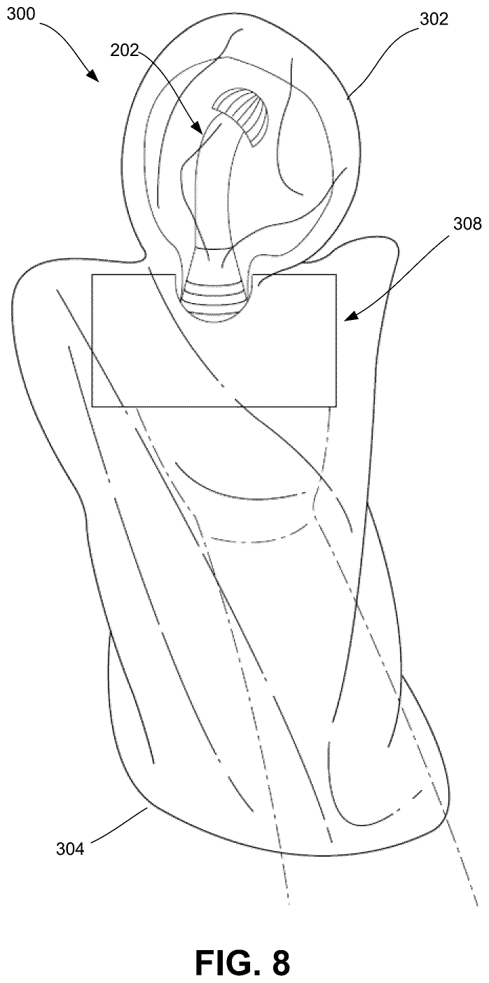

FIG. 8 shows an operator interacting with the measurement assembly, specifically wearing the fingertip probe upon their finger and the disposable barrier placed over the fingertip probe and extending over the operator's hand so as to provide a sterile barrier during the DRE procedure.

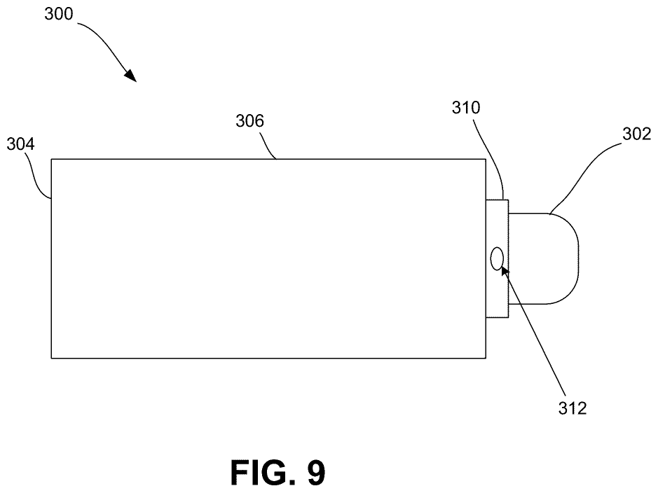

FIG. 9 is a top view of another embodiment of a disposable barrier consistent with the present disclosure. In the illustrated embodiment, the barrier 300 includes a wider proximal opening 304 (i.e., of about 300 mm in diameter) to thereby allow for a greater variety of hand sizes to use the barrier 300. As a result of the wider proximal opening 304, the closed distal end 302 has a particular design to still accommodate and provide a somewhat close fitting to the fingertip probe 202 of the measurement assembly 200. For example, a plastic sheet or band 310 is provided at or near the distal end 302 so as to draw the inner diameter from the sleeve 306 to a tighter diameter as compared to the wider proximal open end 304.

Accordingly, the prostate measurement system of the present invention provides a minimally invasive medical device designed to accurately measure the volume of the prostate gland early in the diagnostic process and throughout a patient's ongoing care with his clinical team. The measurement assembly (i.e., fingertip probe) and disposable barrier are designed for simple, intuitive operation by a urologist, urologic oncologist, urological nurse, or the like (i.e., operation of the device simply requires that the user have a working knowledge of prostate anatomy, of the DRE procedure, and that the user can reliably identify the lateral margins of the prostate gland by palpation). In turn, the prostate measurement system enables early evaluation of prostate health through accurate volumetric measurements, differentiating patients at higher risk and mitigating uncertainty. Patients ultimately benefit from the system of the present invention by avoiding invasive, higher-risk procedures and having better care management, which leads to a better quality of life.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### Incorporation by Reference

For any references and citations to other documents, such as patents, patent applications, patent publications, journals, books, papers, web contents, made throughout this disclosure, all such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A prostate measurement system comprising:
a measurement assembly operably couplable to a control unit and comprising a probe configured to collect measurement data associated with a patient's anatomy during an examination procedure; and
a disposable barrier shaped and/or sized to be removably fitted over the probe and provide a sterile layer of protection between a patient's anatomy and the probe during performance of the examination procedure.

2. The prostate measurement system of claim 1, wherein the barrier comprises:
a closed distal end shaped and/or sized to fully accommodate the probe within; and
an open proximal end shaped and/or sized to allow for the probe and portions of the measurement assembly to pass therethrough and to further accommodate a user's hand.

3. The prostate measurement system of claim 2, wherein the closed distal end comprises an inner diameter that is greater than an outer diameter of the probe.

4. The prostate measurement system of claim 3, wherein the closed distal end is shaped and/or sized to fully accommodate the probe within when an inflatable portion of the probe is fully inflated.

5. The prostate measurement system of claim 2, wherein the closed distal end comprises a first inner diameter of at least 41 mm and a second inner diameter of at least 101 mm.

6. The prostate measurement system of claim 2, wherein the closed distal end comprises a length of between 10 cm and 50 cm.

7. The prostate measurement system of claim 6, wherein the closed distal end comprises a length of between 15 cm and 25 cm.

8. The prostate measurement system of claim 2, wherein the barrier comprises an overall length of between 50 cm and 150 cm.

9. The prostate measurement system of claim 7, wherein the barrier comprises an overall length of between 75 cm and 100 cm.

10. The prostate measurement system of claim 8, wherein the barrier comprises an overall length of approximately 90 cm.

11. The prostate measurement system of claim 1, wherein the barrier comprises a durable material capable of withstanding pressure placed upon the barrier and resisting tearing during performance of the examination procedure.

12. The prostate measurement system of claim 1, wherein the barrier comprises a flexible material allowing for sufficient tactile feedback to be felt by the user when the user is performing the examination procedure with the probe.

13. The prostate measurement system of claim 12, wherein the probe is configured to be mounted to a user's finger and the probe is to be used in a digital rectal examination (DRE) procedure.

14. The prostate measurement system of claim 1, wherein the barrier is single use.

15. The prostate measurement system of claim 1, wherein the barrier comprises a non-stick material allowing for improved and unobstructed insertion and withdrawal of the probe into and out of the interior of the barrier.

16. The prostate measurement system of claim 15, wherein the barrier is comprised of a material comprises a coating provided on an interior surface thereof that exhibits non-stick properties.

17. The prostate measurement system of claim 1, wherein the barrier further comprises an auto-centering feature for properly positioning a user's finger, upon which the probe is to be mounted, into alignment with the distal end of the barrier.

18. The prostate measurement system of claim 17, wherein the auto-centering feature comprises a rigid insert member provided within an interior of the barrier and positioned adjacent to the closed distal end.

19. The prostate measurement system of claim 18, wherein the insert member is shaped and/or sized to maintain separation of opposing interior surfaces of the barrier at or near an entry point into the distal end.

20. The prostate measurement system of claim 19, wherein the insert member comprises a cutout, void, and/or aperture substantially centered with the distal end and is shaped and/or sized to allow for a user's finger and the probe mounted thereto to pass therethrough and into the distal end.
